(19) United States
(12) Patent Application Publication
(10) Pub. No.: US 2009/0111197 A1
(43) Pub. Date: Apr. 30, 2009

(54) HYBRID DEVICE

(75) Inventors: Aman Khan, Bedford (GB); Miles Hugh Eddowes, Cheshire (GB)

Correspondence Address: STEPTOE & JOHNSON LLP, 1330 CONNECTICUT AVENUE, N.W., WASHINGTON, DC, 20036, US

(73) Assignee: Inverness Medical Switzerland GMBH, Zug (CH)

(21) Appl. No.: 11/887,148

(22) PCT Filed: Mar. 28, 2006
(86) PCT No.: PCT/IB2006/002770

§ 371 Date: Apr. 2, 2008

Related U.S. Application Data
(60) Provisional application No. 60/665,863, filed on Mar. 29, 2005.

(51) Int. Cl. G01N 33/536; B01J 19/00

(52) U.S. Cl. 436/536; 422/68.1; 29/428

(57) ABSTRACT

A hybrid device (10) that combines a microfluidic component and a porous components (15) is provided. The microfluidic component includes a sample addition zone (1), a resuspension chamber (13), a mixing chamber (17), and a transfer structure (16), which facilitates fluid flow by capillary action.

### CLAIM OF PRIORITY

This application claims priority under 35 USC §119(e) to U.S. patent application Ser. No. 60/665,863 filed on Mar. 29, 2005, which is incorporated by reference in its entirety.

### TECHNICAL FIELD

This invention relates to devices and methods for performing an assay.

### BACKGROUND

Traditional immunochromatographic or rapid assay tests use polystyrene latex beads loaded with dyes in a dried state near the application point of a fluid sample. Application of the sample then re-suspends and mixes the particles with the sample fluid and analyte contained therein before chromatographic transport moves the sample liquid into the assay zone. A nitrocellulose substrate that is often used also performs the task of mixing at a micro-scale level the sample with the latex via a combination of complex laminar shearing and diffusion.

### SUMMARY

In general, a hybrid device includes a capillary channel fluidly connecting a sample addition zone and a porous carrier.

In one aspect, a hybrid device includes a sample addition zone, a capillary channel having a first end and a second end, the first end being fluidly connected to the sample addition zone, a resuspension chamber connected to the second end of the capillary channel, a transfer structure fluidly connected to the resuspension chamber, and a porous carrier in contact with the transfer structure. Each of the sample addition zone, the capillary channel, the resuspension chamber, the transfer structure, and the porous carrier can be serially connected in fluidic communication within a housing volume.

In another aspect, a method of manufacturing a hybrid device includes providing a microfluidic structure including a sample addition zone, a resuspension chamber, a capillary channel fluidly connecting the sample addition zone and the resuspension chamber, and a transfer structure fluidly connected to the resuspension chamber, and contacting a porous carrier with a portion of the transfer structure.

In another aspect, a method of testing a sample includes applying a sample to a sample addition zone of a device, passing the sample through a capillary channel of the device and into a resuspension chamber of the device to contact an assay reagent, and monitoring a porous carrier of the device for a signal detecting an analyte in the sample. The method can include performing direct fluid transfer from the resuspension chamber to the porous carrier.

In embodiments, the device and methods can include one or more of the following variations.

The capillary channel can include at least one bifurcation. The capillary channel can include a single tapering channel having a bottom plate that tapers out along a direction of fluid flow. The single tapering channel can include a ribbed profile parallel to the direction of fluid flow. The capillary channel can include a ribbed profile. The capillary channel can include at least one wavelet that is perpendicular to a direction of fluid flow.

The sample addition zone can include a urine capture mechanism or a pipette well. The resuspension chamber can include an assay reagent, for example, a dye, an antibody, or an antigen. The resuspension chamber can include a network of posts and stores an assay reagent. The posts can be hexagonal prisms.

A mixing or incubation chamber can be included between the resuspension chamber and the transfer structure. The transfer structure can include a series of substantially parallel channels. The transfer structure can include a series of notches. The porous carrier can be a nitrocellulose membrane.

Certain immunochromatographic tests are not widely used because of their perceived lack of precision compared to analyzers in clinical laboratories. One of the main causes of this lack of precision is the unreliable resuspension of dried assay reagents which are not then evenly distributed in the sample liquid after resuspension and the degree of distribution varies between devices. A hybrid device that contains a microfluidic channel structure coupled to a porous carrier can provide a structure that reliably resuspends dried assay reagents and provides improved precision and reliability in assay readings. The device allows for an even and simultaneous resuspension of assay reagents, thereby minimizing the need for a time gate requirement and improving precision. The device includes a sample addition zone, a resuspension chamber, an optional mixing chamber, and a transfer structure, all serially connected in fluidic communication to improve the mixing of the assay reagent, which is then presented to the porous carrier.

The details of one or more embodiments are set forth in the drawings and description below. Other features, objects, and advantages will be apparent from the description, the drawings, and from the claims.

### BRIEF DESCRIPTION OF THE DRAWINGS

FIG. 1 is a schematic view of a hybrid device.

FIG. 2 is a schematic view of a hybrid device including a series of bifurcations.

FIG. 3 is a schematic view of a portion of a hybrid device.

FIG. 4 is a schematic view of a portion of a hybrid device.

FIG. 5 is a graphic depicting a sequence of video frames of a resuspension chamber filling with a fluorescent solution.

### DETAILED DESCRIPTION

A device for carrying out an assay for a substance in a biological sample includes one or more of the following components: a sample addition zone; a resuspension chamber; a mixing chamber; and a transport structure. A capillary channel fluidly connects two or more of the components of the device. The device can be configured to detect the substance in a sample, such as a biological fluid, for example, urine, plasma or blood. The amount of biological fluid collected from a sample addition zone can be sufficient to perform the assay. The assay can qualitatively or quantitatively identify the presence of the substance in the sample. The assay can be performed at ambient temperature. The assay can be used without specialized equipment and minimal user training. Because of its small size, rapid operation, ease of use and accurate results, the device and assay can be particularly useful for in-home or point-of-care diagnostic tests.

Referring to FIG. 1, a hybrid device 10 includes a capillary channel 13 and a porous carrier 15. When a sample fluid is applied to a sample addition zone 1, fluid can flow into a capillary channel 19 that can include bifurcations 12 and wavelets 18. Each bifurcation forms two fluid paths from a single fluid path. The wavelets can be ribbed channels transverse to the direction of fluid flow in the device. The wavelets can be at the ends of the capillary channel. The capillary can be a tapering channel with grooves or ribs that run parallel to the direction of fluid flow. In general, a hybrid device for testing a biological sample can include distinct zones that form a capillary region, such as a sample addition zone with a tip 11, a resuspension chamber 13, a mixing chamber 17, and a transfer structure 16. Transport structure 16 contacts the porous carrier 15 and transports fluid to the porous carrier.

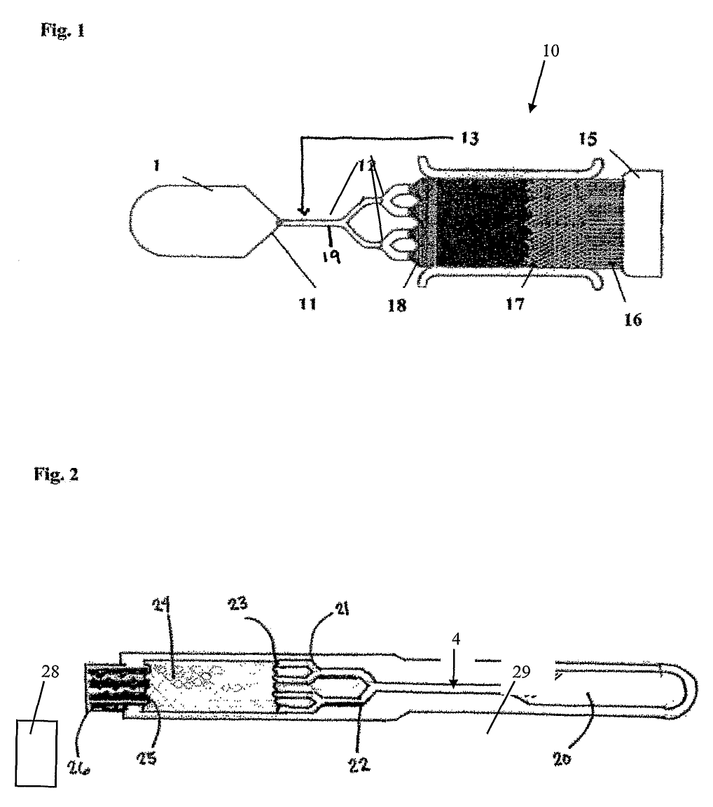

Referring to FIG. 2, an encased hybrid device 4 includes a sample addition zone 20, which can lead to a first bifurcation 22, and a second bifurcation 21. The hybrid device can also include wavelets 23 positioned downstream of the bifurcations. The bifurcations present a united fluid front to the resuspension chamber 24. The hybrid device can include meniscus disrupters 25 that may be positioned, for example, downstream of the resuspension chamber and upstream of a transfer structure 26 that transports fluid to porous carrier 28. The components are encased in housing 29.

Each zone of the hybrid device can have an individual function, and can be serially connected in fluidic communication. Fluidic communication between the sample addition zone and the resuspension chamber can be established by a capillary channel along which fluid can flow.

A capillary channel can be a two walled channel with or without other surface features. The capillary channel can include grooves, wavelets, or combinations thereof.

The capillary channel can be bifurcated. A bifurcation can help to present a more united fluid front to the resuspension chamber, and thereby increase the speed of detection of a substance and improve the accuracy of detected results.

The capillary channel can also be a tapering channel with grooves that run parallel to the direction of fluid flow. The capillary channel can have a first end (or proximal) and a second (or distal) end. The first end can be connected to the sample addition zone, and the second end can be connected to the resuspension chamber. The capillary channel can have at least one bifurcation toward its distal end.

The flow path can be defined, for example, by a depression or depressed region, by fluid channels or capillary action, or by elevations forming walls that can contain a liquid. The fluid can be held on the elevated flow path by surface tension. A microstructured flow path can include capillary structures. In other words, fluid flow along the flow path can be driven and controlled by capillary forces. Fluid flow can also be driven and controlled by mechanical and shear forces over short distances. The sample addition zone can filter or selectively trap components of the sample while allowing other components to flow past the sample preparation region to the remainder of the microfluidic device. Filtered components can be mechanically filtered, chemically or physically bound to a surface, immobilized, or otherwise prevented from traveling to the other regions of the device. The hybrid device can include an assay reagent, such as a chemical dye or an antibody that interacts with the biological sample to produce a detectable change in a reading region of the device.

The sample addition zone can be a rectangular wick, a curved wick, or a pipette well. The sample addition zone can have a sharp notch designed to supply a single capillary with fluid that goes on to feed a resuspension zone.

The hybrid device can include at least one capillary channel to ensure even delivery of the sample from the sample addition zone into the resuspension zone. In one embodiment, the capillary channel can include at least one bifurcation in the channel. The bifurcation can allow the formation of a substantially united fluid front and can reduce or minimize the need for a time gated element to slow fluid flow so that complete mixing takes place. The channel can be positioned along the width of the resuspension chamber. The distance required for the fluid to move from the point of one bifurcation to any point along the width of the resuspension chamber can be substantially equal for all points at which the fluid enters the resuspension chamber. This configuration can allow the resuspension chamber to be fed from all points along its edge substantially simultaneously, and with substantially equal amounts of fluidic resistance to the uptake of fluid.

The capillary channel can also contain a series of ribbed channels or wavelets, perpendicular to the direction of fluid flow. The wavelets can be combined with and positioned at the downstream end of a bifurcation. The wavelets can take the form of shallow notches extending transversely to the liquid flow direction. The wavelets can ensure that an even capillary front is presented to the resuspension chamber.

In another embodiment, the hybrid device can contain a single channel that tapers out along the direction in which the fluid travels from the initial width of the capillary channel to the full width of the resuspension channel. The tapering channel can have a bottom plate with a ribbed profile or grooves that run parallel to the direction of fluid flow. The tapered channel can be used to instead of a bifurcation, or in addition to it.

The capillary channel can generally range in width from 0.01 mm to 0.2 mm, 0.05 mm to 0.15 mm, or 0.08 mm to 0.12 mm, for example. The surfaces of the capillary can be smooth, or have a single groove, or a series of grooves which are parallel or perpendicular to the flow of sample.

The sample addition zone can be designed or shaped with a specific aspect ratio, that makes it suitable for capturing a such as urine, directly from the urine stream. The sample addition zone can also be used to capture a sample by pipetting, or by immersing the sample addition zone in a container that holds a biological sample.

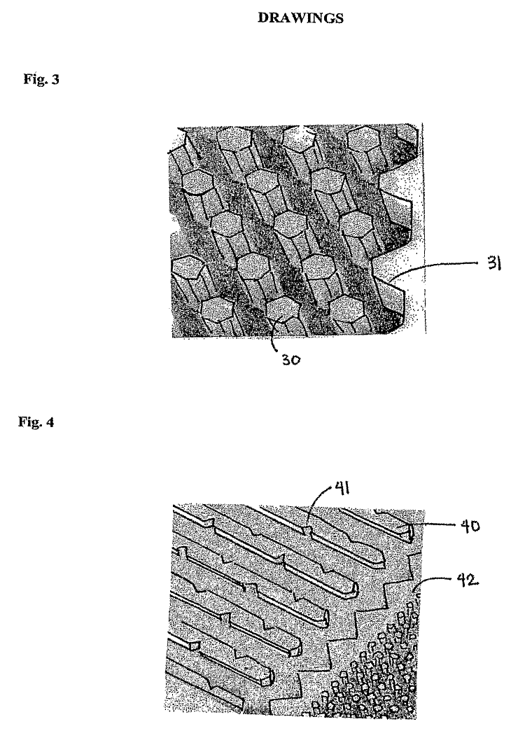

The resuspension chamber can contain closely spaced, geometrically shaped posts, in a substantially regular array which maintains a high capillarity, facilitates simultaneous resuspension of particles, and an even distribution of particles. Referring to FIG. 3, a resuspension chamber of a hybrid device can include posts 30 positioned in an array that is flanked by meniscus disrupters 31 on at least one edge of the resuspension chamber. The posts can have a hexagonal cross-section.

The posts can control both the deposition of dried particles and the flow of shear forces of liquid within the chamber such that the particles can be mixed by the flows of liquid between the posts. Rapid mixing can be achieved by using laminar shear and diffusion over very short distances between the posts. Because the rapid mixing allows the assay reagent to be resuspended at the same time (see Example), there is no need for a time gate in order to quantitatively analyze the sample.

The posts can be designed so that they are relatively small or relatively large, depending on the desired result. For example, smaller posts can provide more even coverage, a more even filling rate with less edge effect, and even mixing. Larger posts can be easier to manufacture, provide faster capillary action, and less drag force for subsequent fluid motion.

The posts can be hexagonal, semi-hexagonal, or any shape that provides a high surface area with high shear forces on the surfaces, while minimizing stagnation areas when in laminar flow, such as is described in WO 96/10747, which is incorporated by reference in its entirety.

The posts can have a specific aspect ratio of 1.5:1, determined by the distance between the posts and the width of each side of the post. For example, the posts may be 90 microns high, with a width of 60 microns spaced center to center 0.104 mm lengthways and 0.120 mm transversely. If a resuspension chamber is approximately 4.5 mm long, 5 mm wide, and 100 microns deep, there can be sufficient space, for example, for 46 posts lengthways and 41 posts transversely.

The relative positions and dimensions of the posts can control both the deposition of dried particles, and the flow and shear forces of a liquid. The positions of the posts can allow diffusion to take place, between the walls of the hexagon posts and the resuspension chamber, and can allow the initial assay reagent to be evenly distributed upon drying. The positions and dimensions of the posts can also ensure that the sample is drawn through the chamber at an appropriate rate to ensure adequate mixing.

The posts can extend over the entire resuspension chamber or over part of the resuspension chamber, both or which are described in U.S. Pat. No. 6,113,855, which is incorporated by reference in its entirety.

The local application of assay reagent in one location of the hybrid device can cause the assay reagent to spread out evenly over the receiving surfaces of the resuspension chamber. The position and distribution of assay reagent within the resuspension zone can help to define the volumes of sample into which the reagents are distributed. Channels at the side of the capillary can help to remove the volumes of liquid that do not contain the desired reagents and can also aid in the lift-off of the assay reagent by creating surges in the velocity of the fluid flow.

The volume of the resuspension chamber, and thereby the reaction mixture, may be any volume which accommodates the reagents and which provides the desired sensitivity of the assay. The shape of the reaction chamber should be such that the movement of the reaction mixture from the reaction chamber is not turbulent and eddies are not formed as a result of the movement out of the reaction chamber. The depth of the reaction chamber should be commensurate with the width of the chamber to accommodate the desired reaction mixture volume. The depth of the reaction chamber can range from about 0.05 mm to 10 mm, for example. To accommodate a particular volume of the reaction chamber, the length and width of the reaction chamber can be adjusted and the depth maintained as narrow as is practical.

At the downstream end of the resuspension chamber, the liquid can down a step to delay fluid progress and assist in developing a uniform fluid front.

A hybrid device can include a mixing or incubation chamber where further mixing and time delay can occur. The incubation chamber can be positioned downstream of the resuspension chamber. It can also include additional posts, which can be designed to be larger, and at a larger pitch, that the posts in the resuspension chamber.

After emerging from the incubation chamber, the liquid can enter parallel paths or channels that are defined between ribs, which are notched to draw up liquid into contact with the detecting porous carrier. The hybrid device can also include a time gate, which is described, for example in U.S. Pat. No. 6,019,944. The time gates can be embedded in membranes or used in devices with membranes.

Once the sample is suspended, it can migrate straight up to the porous carrier, such as a nitrocellulose membrane, through complex paths that aid in the performance of micro-scale mixing through complex laminar shear processes and the continual splitting and recombination of the stream lines as the sample flows around the porous structure. As a result of even distribution, an antibody-antigen binding event can occur almost instantaneously in a substantially homogeneous mixture so that the mixing is as rapid and even as possible.

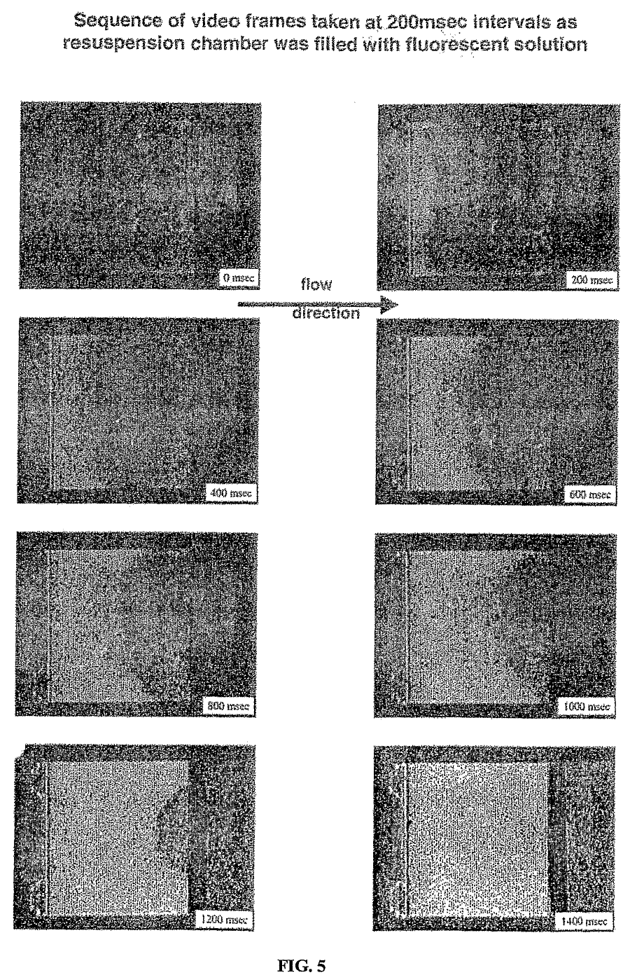

The transfer structure can include an arrangement of small notches that allow the even and immediate transfer of liquid from the transfer structure channels to the pores of a porous carrier, such as a nitrocellulose membrane. The porous carrier can be positioned on top of the transfer structure and can be specifically aligned so as to permit an even uptake of fluid across the capillary profile. Referring to FIG. 4, a transfer structure 41 of a hybrid device can include an arrangement of channels 40 with small notches arranged to permit the transport fluid to the pores of a porous carrier. The transfer structure 41 is adjacent to a resuspension chamber 42, in which an assay reagent contacts the fluid.

A transfer structure can perform direct fluid transfer. Direct fluid transfer is the process of transferring suspended particles to a porous carrier without a time delay or time-gated element. A transfer structure can present the sample, including a suspended assay reagent to a porous carrier by micro-scale mixing through complex laminar-shear processes and the continual splitting and recombination of the stream lines as they flow around its structure. The transfer structure can ensure that an antibody-antigen or other binding event between the sample and a reagent, can occur almost instantaneously.

In one embodiment, the transfer structure can be custom designed to lie flat and parallel to the porous carrier, thereby ensuring a robust fluidic connection. The fluid can pass parallel to and then up into, a flat native surface of the porous carrier. The flat and parallel configuration of the transfer device and porous carrier can cover a large surface area, which can allow a more even fluid uptake even where alignment is not completely precise.

The interface from the resuspension chamber to the transfer structure can include a set of meniscus disrupters that can break the surface tension of the fluid at the interface of the two capillaries or spaces and thereby cause the fluid to move into a capillary or space of lower capillarity. The meniscus disrupters can be used in any part of the hybrid device where fluid must flow from a narrow capillary (high capillarity) to a wider capillary (lower capillarity).

Nitrocellulose has considerable advantage over conventional carrier materials, such as paper, because it has a natural ability to bind proteins without requiring prior sensitization. Specific binding reagents, such as immunoglobulins, can be applied directly to nitrocellulose and immobilized thereon. No chemical treatment is required which can interfere with the essential specific binding activity of the reagent. Unused binding sites on the nitrocellulose can thereafter be blocked using simple materials, such as polyvinyl alcohol. Moreover, nitrocellulose is readily available in a range of pore sizes and this facilitates the selection of a carrier material to suit particularly requirements such as sample flow rate.

The assay reagent can bind to or react with the analyte to produce a detectable change. The detectable change can be, for example, a change in optical properties such as an absorption or emission of light. The detectable change can be a change in color. The reagent can include an affinity molecule that binds to the analyte. The affinity molecule preferably binds tightly and specifically to the analyte. In other words, the affinity molecule has a large association constant for the analyte, while having a much lower (for example, by one or more orders of magnitude) association constants for other components present in the liquid sample. The affinity molecule can be, for example, a protein, a peptide, an antibody, a nucleic acid, or a small molecule. The analyte can be, for example, a protein, a peptide, an antibody, a nucleic acid, or a small molecule. The affinity molecule can be selected for its affinity and selectivity towards its ligand. The assay reagent can include a dye, for example, colored latex, or a particle, for example, a nanoparticle, including colloidal gold particles.

An assay reagent can also be configured to perform a semi-quantitative or quantitative assay, as for example, is described in Clinical Chemistry (1993) 39, 619-624, which is incorporated by reference in its entirety. This format utilizes a competitive binding of antigen and antigen label along a solid phase carrier. The improvement is that the use of the diagnostic element described herein for the above cited method would require a smaller sample volume and improved binding efficiency to the solid phase surface.

The assay reagent, such as nanoparticles or latex particles coated with receptors, can be applied to surfaces of many types of immuno-assay devices, as for example, to “dipsticks,” as described in U.S. Pat. No. 6,019,944. Dipsticks are generally used as a solid phase onto which are bound, as a result of the assay process, for example, the ligand receptor conjugate. Dipsticks can generally incorporate membranes; however, a disadvantage in the use of membranes in dipsticks is the difficulty in washing the unbound ligand receptor from the membrane. Thus, an improvement in the use of dipsticks can be to immobilize receptor coated latex or nanoparticles directly onto a plastic surface of the dipstick. The removal of unbound ligand conjugate from the plastic surface is thus more efficient than removal from a membrane.

An assay based on the above principles can be used to determine a wide variety of analytes by choice of appropriate specific binding reagents. The analytes can be, for example, proteins, haptens, immunoglobulins, hormones, polynucleotides, steroids, drugs, infectious disease agents (e.g. of bacterial or vital origin) such as Streptococcus, Neisseria and Chlamydia. Sandwich assays, for example, may be performed for analytes such as hCG, LH, and infectious disease agents, whereas competition assays, for example, may be carried out for analytes such as E-3-G (estrone-3-glucoronide) and P-3-G (progesterone-3-glucuronide).

The hybrid device microfluidic and porous carrier components can be made according to the principles and descriptions contained in U.S. Pat. Nos. 5,656,503, 5,885,520, 6,019,944, 6,156,270, and 6,113,855, each of which is incorporated by reference in its entirety.

The hybrid device can be composed of polycarbonate. It can be formed by laser ablation of a polycarbonate surface followed by a hydrophilic treatment, such as plasma co-polymerization. The device can be made using an injection molding process with a mold that has been made using electroplating using a lithography technique. The top plate of the device can be a hot melt polymer film with a heat activated adhesive chosen to be compatible with long term storage of the finished assay device.

Generally, the hybrid device can have a thickness of approximately 2 mm to 20 mm, lengths of about 3 cm to 10 cm, and widths of about 1 cm to 4 cm. The dimensions may be adjusted depending on the particular purpose of the assay.

The hybrid device can be made with a plastic, elastomer, latex, silicon, or metal. The elastomer can comprise polyethylene, polypropylene, polystyrene, polyacrylates, or latex. Components of the device can be prepared from latex, polystyrene latex, or hydrophobic polymers, TEFLON®, or polycarbonate, such as is described in WO 98/43739, which is incorporated by reference in its entirety.

### EXAMPLE

Experiments demonstrated a rapid filling and even resuspension of latex particles with a hybrid device. A device depicted in FIG. 1 was constructed that had the following characteristics. The resuspension chamber was 4.5 mm long, 5 mm wide and 100 microns deep. It had evenly spaced hexagonal pillars 90 microns high with a width of 60 microns spaced center to center 0.104 mm lengthways and 0.120 mm transversely. There were 46 posts in a row lengthways and 41 transversely. Fluorescent latex (green fluorescence, diameter approximately 0.5 micron) was dried into the resuspension chamber by pipetting approximately 1.5 microliters of a 2% w/v suspension of water onto the surface of the resuspension chamber. This suspension spread itself evenly over the chamber and was then allowed to air dry. A lid was added by applying adhesive coated laminae from the sample application zone to the area exposed. A 50 microliter aliquot of water was applied to the sample application zone and then traveled through the device by capillary flow. The process was filmed using a video camera under illumination suitable for exciting the fluorescence of the latex. The dry resuspension chamber (0 msec) showed a very small amount of fluorescence but as the latex became wet the observed fluorescence becomes much greater due to more favorable optical conditions. The spread of liquid across the chamber is easily seen through the subsequent video frames taken at 200 msec intervals. By 1400 msec, the chamber is clearly filled. The video frames are shown in FIG. 5. Additional observations using higher magnification showed that the latex was also resuspended at the same time and appeared evenly distributed within the resuspension chamber.

Other embodiments are within the scope of the following claims.

What is claimed is:

1. A hybrid device comprising:
a sample addition zone;
a capillary channel having a first end and a second end, the first end being fluidly connected to the sample addition zone;
a resuspension chamber connected to the second end of the capillary channel;
a transfer structure fluidly connected to the resuspension chamber; and
a porous carrier in contact with the transfer structure.

2. The device of claim 1, wherein the capillary channel includes at least one bifurcation.

3. The device of claim 1, wherein the capillary channel includes a single tapering channel having a bottom plate that tapers out along a direction of fluid flow.

4. The device of claim 3, wherein the single tapering channel includes a ribbed profile parallel to the direction of fluid flow.

5. The device of claim 1, wherein the capillary channel includes a ribbed profile.

6. The device of claim 1, wherein the capillary channel includes at least one wavelet that is perpendicular to a direction of fluid flow.

7. The device of claim 1, wherein the sample addition zone includes a urine capture mechanism or a pipette well.

8. The device of claim 1, wherein the resuspension chamber includes an assay reagent.

9. The device of claim 1, wherein the resuspension chamber includes a network of posts and an assay reagent.

10. The device of claim 8, wherein the posts are hexagonal prisms.

11. The device of claim 1, further comprising a mixing or incubation chamber between the resuspension chamber and the transfer structure.

12. The device of claim 1, wherein the transfer structure includes a series of substantially parallel channels.

13. The device of claim 1, wherein the transfer structure includes a series of notches.

14. The device of claim 1, wherein the porous carrier is a nitrocellulose membrane.

15. A method of manufacturing a hybrid device comprising:
providing a microfluidic structure including a sample addition zone, a resuspension chamber, a capillary channel fluidly connecting the sample addition zone and the resuspension chamber, and a transfer structure fluidly connected to the resuspension chamber; and
contacting a porous carrier with a portion of the transfer structure.

16. The method of claim 15, wherein the capillary channel includes at least one bifurcation.

17. The method of claim 15, wherein the capillary channel includes a single tapering channel having a bottom plate that tapers out along a direction of fluid flow.

18. The method of claim 17, wherein the single tapering channel includes a ribbed profile parallel to the direction of fluid flow.

19. The method of claim 15, wherein the capillary channel includes a ribbed profile.

20. The method of claim 15, wherein the capillary channel includes at least one wavelet that is perpendicular to a direction of fluid flow.

21. The method of claim 15, wherein the sample addition zone includes a urine capture mechanism or a pipette well.

22. The method of claim 15, wherein the resuspension chamber includes an assay reagent.

23. The method of claim 15, wherein the resuspension chamber includes a network of posts and includes an assay reagent.

24. The method of claim 22, wherein the posts are hexagonal prisms.

25. The method of claim 15, further comprising a mixing or incubation chamber between the resuspension chamber and the transfer structure.

26. The method of claim 15, wherein the transfer structure includes a series of substantially parallel channels.

27. The method of claim 5, wherein the transfer structure includes a series of notches.

28. The method of claim 1, wherein the porous carrier is a nitrocellulose membrane.

29. A method of testing a sample in a hybrid device comprising:
applying a sample to a sample addition zone of a device;
passing the sample through a capillary channel of the device and into a resuspension chamber of the device to contact an assay reagent; and
monitoring a porous carrier for a signal detecting an analyte in the sample.

30. The method of claim 29, further comprising performing direct fluid transfer from the resuspension chamber to the porous carrier.
